(19) United States
(12) Patent Application Publication
(10) Pub. No.: US 2020/0386730 A1
(43) Pub. Date: Dec. 10, 2020

(54) AUTOMATED SYSTEM FOR DETECTING BACTERIA IMPLICATED IN INFECTIONS OR DISEASES USING A MULTISENSOR SYSTEM INCORPORATING AN OLFACTOMETRY DEVICE RECOGNISING THE RELEASED METABOLITES

(71) Applicant: Nassira LACHLAK, Toulouse (FR)

(72) Inventor: Nassira LACHLAK, Toulouse (FR)

(21) Appl. No.: 16/787,073

(22) Filed: Feb. 11, 2020

Related U.S. Application Data
(63) Continuation-in-part of application No. PCT/DZ2018/050004, filed on Aug. 29, 2018.

(30) Foreign Application Priority Data
Oct. 9, 2017 (DZ) 170565

(51) Int. Cl. G01N 33/00

(52) U.S. Cl. CPC G01N 33/0031; G01N 33/0047

(57) ABSTRACT

The invention provides a rapid medical diagnosis through the use of the electronic nose, thanks to the volatile organic compounds released by the bacteria incriminated in an infection.

The use of conventional bacteriology takes 2-3 days to give a diagnosis to the biologist of the type of bacteria incriminated in the infection.

From a multi-sensor system, volatile organic compounds (VOCs) e.g. metal oxides are detected.

The sensors used are CO2, amines, and heptanone4, in case of infections and metabolic pathologies. The bacterial species incriminated in the infection is detected from the VOCs released by the bacteria in 30 minutes.

The use of biosensors was chosen for the development of a portable system for a small sample or fixed device to analyze large quantities of samples in medical facilities. This makes it possible to optimize the size and consumption. The results of these biosensors are generally reproducible and fast in detection.

### DOMAIN TO WHICH THE INVENTION RELATES

Analysis of samples from symptomatic patients: Detection and prevention of diseases, infections and other pathologies in the gynecological field by a multi-sensor system.

The invention relates to a design of an automaton enabling the analysis by sensors of certain infections or pathologies in the field of gynecology and other pathologies by this multi-sensor system called medical Olfaction.

It can also be used for the detection of nosocomial infections, the detection of diabetes and other agri-food and environmental applications.

### STATE OF THE PREVIOUS CONCEPT

To date, biomedical applications are still unexploited in certain areas, especially in gynecology, nosocomial pneumopathies and other pathologies. This multi-sensor system called the Medical Olfactometer lends itself well to these biological assays, since it allows the analysis of samples in a complex biological medium, without resorting to chemical reagents just by olfactometry. The design of this multisensor system is intended for medical analysis laboratories.

This multi-sensor system also validates pathologies in other areas of health as well as in the agri-food field and can even be used in the environment

### OBJECT OF THE INVENTION

The design of this multisensor system is a method of characterization of volatile organic metabolites for a practical, quick and easy diagnosis. The use of these sensors in these samples that release VOC generate characterization data from the output signals from these sensors. The set of sensors is placed in a room. The volatile organic metabolites are transported to the sensor chamber through a flexible vinyl tube connected to the sniffer head and placed just above the sample to be analyzed. The output signals from the various sensors installed in the chamber are normalized with respect to values determined in a prior calibration operation during which the sniffer head has made it possible to record the data of at least one reference sample. From the output signals from the gas sensors, the multi-sensor system must enable the biologist and the practitioner to deal with new and emerging aspects of infections or pathologies or metabolic diseases such as diabetes, nosocomial pneumopathies and many other diseases affecting a population at any age.

The present invention relates to the creation of an automaton for medical analysis laboratories having a high resolution capable of quickly and reliably diagnosing a sample in a patient, by the development of sensors specific to the molecules released by the bacteria incriminated in infections or diseases.

The use of sensors of different types of technologies makes it possible to give a specific signature to the bacterium incriminated in the pathology.

It is necessary to study beforehand the biochemical characteristics of the bacterium by means of gas chromatography in order to determine the sensors to use.

The new technology of this multi-sensor system is considered to be a fast tool for identifying volatile organic compounds. The extension of its use to medical clinical diagnosis is now being considered. This is the object of this invention.

The biosensor lends itself well to these assays, since it allows the analysis of a sample in a complex biological environment, without resorting to chemical reagents.

In gynecology, the prevalence of bacterial infections among our population of consultants shows a significant number of symptomatic patients.

The advantage of the present invention lies in its high resolution and discrimination capabilities achieved through the use of signals from several sensors integrated into the chamber

### 1.5 STATEMENT OF FIGURES

The invention will be better understood from the description which follows, with reference to the appended graphs of this method.

FIG. 1: The conical chamber that carries the sensors.

FIG. 2: The kinetics and olfactory impressions of two samples in tests.

FIG. 3: Comparison of the olfactory impressions of two samples carrying vaginosis, with a similarity index of 37% In order to compare the repeatability of the method, the injections of two different vials but of the same sample are compared.

FIG. 3: Excellent discrimination is achieved at 99%.

FIG. 4: The system design of the electronic board with sensor ports.

FIG. 5: The acquisition card of the data collected by the sensors.

### PRESENTATION OF THE INVENTION

Chemical sensor systems enable the detection of volatile organic compounds (VOCs) for clinical diagnostics. This new approach to medical diagnosis offers exciting opportunities in many medical areas.

The challenge of medical applications is indeed to be able to overcome the great variability of patients. The technology seems promising to relieve specialists working in clinics to carry out screening and diagnosis on the population as a whole. Improving the rate of disease detection and prevention in the population would significantly reduce the indirect costs of late diagnosis. The goal is the realization of a multi-sensor system for detecting these pathologies according to the concentration of the molecule detected. This will make it possible to treat the type of infection on the spot before its evolution and will avoid further examinations in the field of health. Many medical applications will be considered. The same device can respond to several medical applications to diagnose early diseases. The application of this multi sensor system can be considered in the agri-food and environmental field.

### Model of Realization of the Invention

The method relates to the production of a chamber equipped with several sensors that make it possible to have a fingerprint characterizing the specific biochemistry of the bacterium involved in the infection or the pathology by comparing it to a learning base or database in order to detect or identify the bacterium incriminated in this pathology. This learning base or database is established during the learning phase of the system. The sensor chamber is made by assembling two cones by their bases. The sensors are placed between the two cones and are directed downwards. For each round shaped sensor a diameter of 2 cm is reserved whereas for rectangular sensors a surface of 5.2 cm by 1.7 cm is reserved. This will require a total circular area of 11 cm diameter to house the 12 sensors used. The following types of sensors were used for the realization of the automaton:

TABLE

| | Gaz cible |
|---|---|
| Sensor 1 | Contaminants Air (hydrogen, Ethanol, etc.) |
| Sensor 2 | Contaminants Air (VOCs, Ammonia, H₂S, etc.) |
| Sensor 3 | Alcool, Solvant vapors |
| Sensor 4 | Methane, Propane, and Butane |
| Sensor 5 | CO |
| Sensor 6 | CO₂ |
| Sensor 7 | Trémythylamine |
| Sensor 8 | Amonia |

sensors in a closed environment. The shape of the chamber is conical and is teflon with 12 sensors.

It has an inlet to receive VOCs and a VOC release outlet through a pump. These sensors are used primarily to test the chamber and study the behavior that allows the aspiration of VOC samples and their expiration from the inside of the room, outward.

Injection and extraction of VOCs are automated by this multisensor system to analyze the samples see FIG. 1.

The volatile organic compounds released by the microorganisms at 37° C. are automatically drawn from the sample, for a period of time, through the sniffer head with this pump.

Specific sensors installed in the chamber are sensitive to volatile organic compounds that are captured.

The acquisition of the data from the sensors is provided by the interface card which allows among other things the polarization and the heating of the sensors.

The data from the sample analysis is sent via a data mining program, installed on an embedded system board.

The results are graphically represented in real time and finally stored in files for later statistical study

The studies carried out have shown that the release of VOC is optimal when the samples to be analyzed are at a temperature close to that of the human body (37° C.). Thus the system in place is equipped with a water bath with automatic regulation of the temperature following the instruction introduced by the user. This water bath is fixed on a plateau carrying the tubes of the samples of analysis.

To start the analysis it is necessary to ensure reactivity of the sensor chamber, the polarization card, heating of the samples to 37° C. (ideal temperature for the release of volatile compounds of bacteria).

The sniffer head draws volatile organic compounds from the samples and then transports them to the analysis chamber which is equipped with sensors.

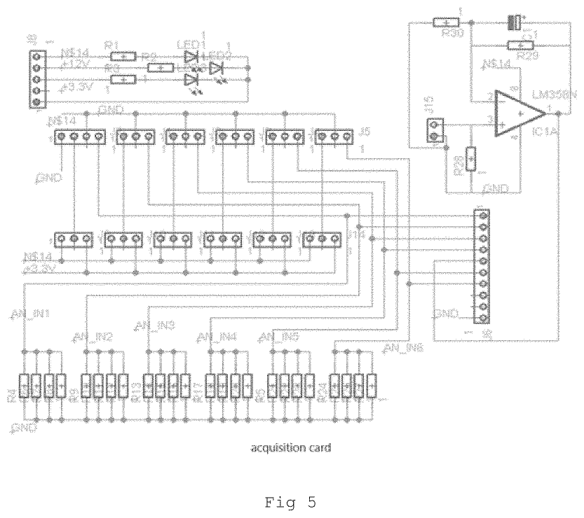

The electronic circuit that provides power to the system is summarized in FIG. 4 below.

The polarization and heating circuits of the sensors allow normal operation of the sensors.

The bias voltage of the VC sensors is between 0 and 5 V. This voltage is identical for all the sensors of the matrix. The sensors are heated either at constant temperatures (isothermal mode) or by a temperature modulation.

The circuit made for a power supply makes it possible to adjust the heating and polarization voltages as shown in FIG. 4 and FIG. 5.

The RS resistance of the sensor is deduced from the measurement of the voltage VRL across the load resistor RL (10 kW):

RS=((VC/VRL)−1)×RL

The number of sensors to put together is defined during this stage of the design of the acquisition card connected to the board of the embedded system. The shaping circuit portion “isothermal regime” is closed.

The acquisition card is connected to an embedded motherboard that allows the data to be processed and represented as graphs. The capture card converts the analog sensor data into numerical values. The acquisition card also manages all peripherals: heating resistor pump and others. This acquisition card dialogues with the embedded motherboard to receive commands to execute and send useful information for the smooth running of the analysis process. Each sample is analyzed according to a well-defined cycle. The cycle begins with a 30 second VOC suction phase, then a data measurement phase and ends with a chamber emptying phase. All phases are configurable to ensure reliable operation for most VOCs to be detected.

Once the optimization is done by each sensor sensitive to volatile organic compounds specific to the bacteria incriminated in these infections or diseases. The detection of the volatile organic compounds of each vaginal exudate is studied according to the technique provided in my previous patent by a manipulator arm that sucks with a sniffing head the volatile organic compounds released by the bacteria of these vaginal samples to determine the pathology. The intensity of the output signals from the sensors depends on the concentration of bacteria in the assay sample. In fact, the introduction of this multi-sensor system makes it possible to ensure the arrival of gases, in an equal manner, to all the existing sensors.

The cleaning of the chamber (emptying) after each passage of a sample is carried out in 5 min the time required for aspiration of the gases released by the bacteria of the vaginal flora (vaginal exudates). The model of realization of the invention can be described as follows: The assay is run in the crude state without adding reagents to the sample, which is divided into two 2 ml samples in each vial so that the repetition of the method can be tested. These samples are analyzed by this multi-sensor system. After the acquisition of the data, a comparison between the responses of the sensors for the different samples was made.

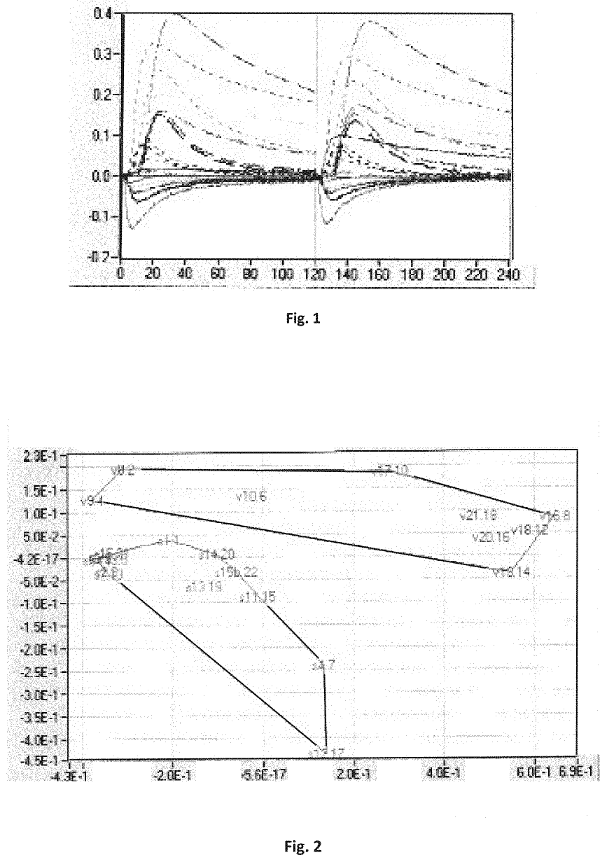

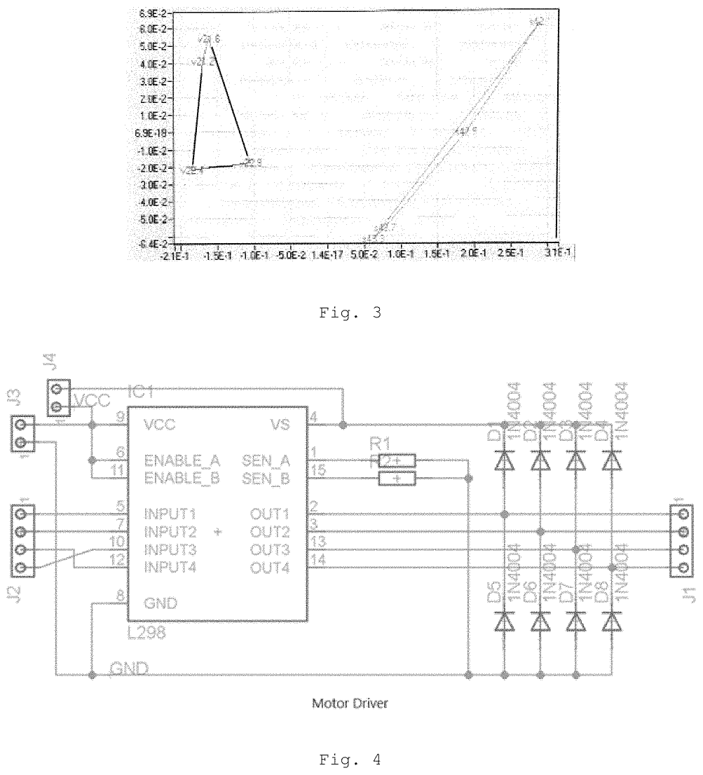

It is important to note that an intense response does not correspond to a strong smell and vice versa. The intensity given by this multi-sensor system must be correlated with the sensory panel scores or the results of a chemical analysis for data processing by statistical methods. The responses of the sensors in place are represented by the kinetics and olfactory impressions of two samples in FIG. 2. The difference between the injection of the same sample is greater than or equal to the difference between a healthy sample and a carrier sample. The headspace generation temperature and the headspace generation time will be increased to improve repeatability for the same sample and two different bottles. Principal component analysis makes it possible to present the information given by the various sensors mentioned above in two axes. 93.46% of the information is represented on the X axis and 5.32% on the Y axis. 98.78% of the total information given. by these sensors which is represented in FIG. 3.

Each color represents a sample: healthy sample in red and sample carrying vaginosis (bacteria incriminated Gardnerella vaginalis) in blue. The samples undergo a repeat of analysis. The samples whose result is the same are grouped together. The method used thus makes it possible to obtain a well confirmed result. There is an intersection between healthy samples and carrier samples. After analysis, the VOCs released by the samples are subjected to a confirmatory test by adding a drop of KOH which allows the release of the amines confirming the vaginosis by sniffing the samples. The principal components analysis (PCA) allows the information given by the selected sensors to be graphically represented on two axes. 93.33% of the information is represented on the X axis and 6.58% on the Y axis; 99.91% of the total information given by the sensors is represented in FIG. 3. Each color represents a sample: healthy sample (in red) and samples carrying vaginosis (in blue). Excellent discrimination is obtained between healthy samples and samples carrying the infection. Despite the variability obtained for each group, the two classes of samples (carriers of the disease and healthy are differentiated.

1. The automaton comprises a multi-sensor system which detects released volatile molecules incriminated in infections, diseases and other odors which are previously studied by an olfactometer provided with several different sensors located in a chamber.

2. The multi-sensor system according to claim 1, wherein the multi-sensor system is characterized by the manufacture of a conical shaped Teflon chamber comprising a plurality of sensors and the system has an automated autosampler that carries a water bath with a temperature adjustable according to the growth of each bacteria in the environment and the temperature control is managed by the motherboard.

3. This system with a chamber according to claim 1 wherein the system comprises an inlet and an outlet in the chamber for conveying the volatile organic compounds and the circulation of gases is ensured by an automated pump.

4. The multi-sensor system according to claim 1, wherein the multi-sensor system is characterized by the manufacture of a Teflon chamber of 4 and the multisensor system according to claim 3 comprises the use of a sampling head carried by a manipulator arm which sniffs the molecules released by the microorganisms in the sample of the sample and this manipulator arm moves in the direction of the X, Y and Z axes to sniff out the volatile organic compounds released, then they are sent into the chamber of the sensors specific to these volatile organic compounds.

5. The automaton comprising the multi-sensor system according to claim 1, wherein the automation is provided with a bias voltage for each VC sensor between 0 and 5 volts, and this voltage is identical for all the sensors of the matrix and the sensors are heated either at constant temperature (isothermal mode) or by a temperature modulation according to the samples taken and the analysis to be done.

6. The multi-sensor system according, to claim 1, wherein the multi-sensor system comprises the establishment of the polarization card, with an ideal temperature for the release of volatile organic compounds by the bacteria in the sample flasks.

7. The multi-sensor system according to claim 1, wherein a procedure for detecting infections or diseases in the gynecological field from the volatile organic compounds of the doderlein flora is characterized by the multi-sensor system for identifying these VOCs of this flora.

8. The multi-sensor system according to claim 5, wherein the analysis of the samples is directly on the collection of exudates without adding any chemical reagent or molecule for the release of volatile compounds of the bacteria incriminated in the infection or disease.

9. The multi-sensor system according to claim 1, wherein an automatic sample changer according to claim 1 is placed in a water bath at a suitable temperature programmed according to the bacterial growth conditions.

10. The multi-sensor system according to claim 5, wherein the samples are analyzed at least twice to give good reproducibility and reliability of results. This analysis is done by signal processing to classify symptomatic patients on one side and asymptomatic patients on the other.

11. The procedure for detecting infections according to claim 7, wherein the procedure for detecting infections or diseases in the gynecological field is based on the VOCs of the vaginal exudates which are stored in a data base of this system
and each graph corresponds to a specific response of a given sensor, allowing the discrimination of the samples and Principal Components Analysis (PCA) is performed on the data
and the analysis processing is characterized by signals following a statistical analysis and a processing operation by neural network compared to a classification established during the learning phase of a sample taken in the course of analysis.

12. The multi-sensor system according to claim 5, wherein an olfactometry automaton housing the multi-sensor system, comprises a chamber cleaning pump which collects the volatile organic compounds.

13. The multi-sensor system according to claim 5, wherein the system allows the detection of the bacteria incriminated in infections or diseases, in the gynecological field, from release of VOC in vaginal exudates.

14. The multi-sensor system according to claim 13, wherein the release of these VOC is activated by a temperature according to claims 2 and 6, which is characterized by the principal component analysis (PCA), for presenting the information given by the sensors on two axes graphically and this automaton can be used even in diabetology, agrifood and environment.

15. The automaton comprising the multi-sensor system according to claim 1, where in the automation is equipped with this VOC detection system is characterized by the samples of the same sample which are close to each other,
and the principal component analysis makes it possible to graphically represent the information given by the sensors on two axes and
88.68% of the information is represented on the X axis and 7.01% on the Y axis and 95.69% of the total information given by the sensors,
and each color specifically represents the asymptomatic patient or the symptomatic patient.

16. The procedure for detecting infections according to claim 7, wherein the procedure for detecting infections or diseases in the gynecological field from the volatile organic compounds of vaginal exudates according to claim 1, characterized by an excellent discrimination obtained at 99.98% with the CO2 sensor and since 2 ml samples for the analysis series is taken which makes it possible to homogenize and standardize the quality of sample analysis.
